(19) United States
(12) Patent Application Publication
(10) Pub. No.: US 2018/0250284 A1
(43) Pub. Date: Sep. 6, 2018

(54) QUINOLINE DERIVATIVES FOR USE IN TREATING LEUKODYSTROPHY AND TREATMENT METHOD

(71) Applicants: GEORG-AUGUST-UNIVERSITÄT GÖTTINGEN STIFTUNG ÖFFENTLICHEN RECHTS, UNIVERSITÄTSMEDIZIN, Göttingen (DE); TEVA PHARMACEUTICAL INDUSTRIES LTD., Petach Tikva (IL)

(72) Inventors: Volker KNAPPERTZ, Potomac, MD (US); Michael HAYDEN, Vancouver (CA); Wolfgang BRÜCK, Rosdorf (DE); Stefan NESSLER, Göttingen (DE)

(73) Assignees: Georg-August-Universität Göttingen Stiftung Öffentlichen Rechts, Universitätsmedizin, Göttingen (DE); Teva Pharmaceutical Industries Ltd., Petach Tikva (IL)

(21) Appl. No.: 15/758,098

(22) PCT Filed: Sep. 8, 2016
(86) PCT No.: PCT/EP2016/071181

§ 371 Date: Mar. 7, 2018

Related U.S. Application Data
(60) Provisional application No. 62/215,619, filed on Sep. 8, 2015.

(30) Foreign Application Priority Data
Sep. 9, 2015 (EP) 15184456.0

(51) Int. Cl. A61K 31/4704; A61K 9/00

(52) U.S. Cl. CPC A61K 31/4704; A61K 9/0053

(57) ABSTRACT

The present invention relates in a first aspect to compounds for use in the treatment of leukodystrophy whereby these compounds are quinoline derivatives, e.g. laquinimod. In a further aspect, the present invention relates to methods for the treatment of Leukodystrophy, in particular, peroxisomal disorders including Zellweger syndrome.

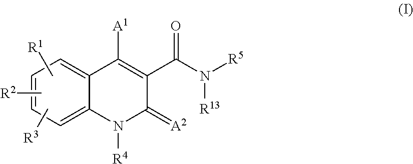

The present invention relates in a first aspect to compounds for use in the treatment of leukodystrophy whereby these compounds are quinoline derivatives, e.g. laquinimod. In a further aspect, the present invention relates to methods for the treatment of leukodystrophy, in particular, peroxisomal disorders including Zellweger syndrome.

### PRIOR ART

Of note, throughout this application, various publications are referred to by the author and year of publication. Full citations for these publications are presented in a References section immediately before the claims. Disclosures of the publications cited in the References section in their entireties are hereby incorporated by reference into this application in order to more fully describe the state of the art as of the date of the invention described herein.

Leukodystrophies are a heterogeneous group of metabolic and/or hereditary central nervous system (CNS) disorders with defects in cell metabolism, leading to characteristic pathological changes in the CNS white matter. Leukodystrophies are disorders that result in white matter abnormalities in the central nervous system (Parikh at al., 2015). These disorders are individually rare but collectively, they have an incidence of 1 in 7000 (Holman at al., 2015). In addition, leukodystrophies are difficult to diagnose, and often remain undiagnosed or misdiagnosed (Parikh at al., 2015).

Early clinical symptoms of leukodystrophies most commonly involve motor symptoms, manifesting as delayed development of motor skills, a plateau in development of motor skills, or regression in motor skills, resulting in rigidity, dystonia, ataxia, and bulbar symptoms (Parikh at al., 2015).

In more advanced stages of most leukodystrophies, cognitive impairment (ex. developmental delay and intellectual disability) will become apparent as myelin disturbance causes neuronal and axonal dysfunction. In some patients, cognitive impairment will progress to dementia (Parikh et al., 2015).

Other neurologic features include nystagmus, irritability, titubation, autonomic dysfunction, and encephalopathy. Some extraneurologic features include endocrine dysfunction, ophthalmologic abnormalities, cortical visual impairment, dental abnormalities, dysmorphic physical features, tendinous xanthomas, skeletal abnormalities, bony abnormalities, hearing impairment, hepatosplenomegaly, cutaneous abnormalities, ovarian dysgenesis or gastrointestinal symptoms (Parikh et al., 2015).

Leukodystrophies are symptomatically treatable and require thorough management by the caregiver and responsible clinician to address the complex array of symptoms. For certain forms of leukodystrophies, hematopoietic stem cell therapy “may” be an available treatment. In addition, a number of disease-specific therapies are currently in or on the verge of human trials (Helman et al., 2015).

Among them are peroxisomal disorders characterized by either a failure of organelle formation (peroxisome biogenesis disorders) or a defect in a single peroxisomal protein or a distinct peroxisomal pathway (Aubourg and Wanders, 2013). Peroxisome biogenesis disorders are caused by defects in PEX genes that encode peroxins required for the normal biogenesis of peroxisome (Crane D. I., 2014).

Peroxisomes are organelles that are present in virtually all cell types and play an important role in the detoxification of reactive oxygen species, synthesis of plasmalogens, α- or β-oxidation of fatty acids, specifically very long chain fatty acids (VLCFA). (Bottelbergs et al., 2010; Kassmann et al., 2007). Mice lacking functional peroxisomes in the brain exhibit severe neurological problems, including motoric and coordination deficits and cognitive impairment (Bottelbergs et al., 2010).

PBDs are inherited in an autosomal recessive manner (Crane D. I., 2014). There are two groups of PBD: 1) the Zellweger syndrome spectrum, which result from defects in the peroxins required for membrane protein import, and 2) rhizomelic chondrodysplasia punctate (RCDP), which result from defects in the import of matrix proteins. (Steinberg et al., 2006; Crane D. I., 2014).

The Zellweger syndrome spectrum includes Zellweger syndrome (ZS), neonatal adrenoleukodystrophy (NALD), and infantile Refsum disease (IRD). ZS is the most severe, and IRD is the least severe. Disease severity is related to the nature of the PEX gene mutation and the resulting impact on the function of the affected peroxin. ZS patients rarely survive their first year, whereas IRD patients may survive beyond their third decade (Crane D. I., 2014).

Symptoms of ZS include craniofacial abnormalities (ex. high forehead, hypoplastic supraorbital ridges, epicanthal folds, midface hypoplasia, and large anterior fontanel), eye abnormalities (ex. corneal clouding, cataracts, flaucoma, optic atrophy, and retinal anomalies), neuronal migration defects (ex. polymicrogyria, Purkinje cell heterotopia, olivary nucleus abnormities), hepatomegaly, renal cysts, chondrodysplasia punctate, etc. Affected children also present profound hypotonia, seizures, and inability to feed. There is also an absence of neonatal and deep tendon reflexes and little spontaneous movement (Steinberg et al., 2006).

Symptoms of NALD and IRD include craniofacial abnormalities, hypotonia, seizures, spasticity, sensorineural hearing loss, retinitis pigmentosa, etc. However, the presentation of these symptoms are milder than in ZS (Steinberg et al., 2006).

Symptoms of RCDP include disturbed ossification (ex. shortening of the proximal long bones with metaphyseal cupping coronal clefts of the vertebral bodies, epiphyseal stippling), contractures, bilateral cataracts, abnormal faces with frontal bossing, depressed nasal bridge, small nose, ichthyosis, central nervous system abnormalities (ex. cerebral and cerebellar atrophy, abnormalities of myelination, neuronal migration defects), growth retardation, psychomotor retardation, respiratory complications, congenital cataracts, chondrodysplasia, rhizomelia, mental deficiency, growth deficiency, and learning disability.

That is, peroxisomes are cell organelles present in most eukaryotic cells, which contain more than 50 enzymes catalyzing anabolic and catabolic reactions. Among others, peroxisomes are involved in the biosynthesis of ether lipids and in α- and β-oxidation pathways (Wanders and Poll-The, 2015).

The proteins required for peroxisome biogenesis are named Pex proteins/peroxins and encoded by PEX genes and up to now 16 human peroxins have been described. The prototypic peroxisomal biogenesis disorder is the Zellweger syndrome or Zellweger syndrome spectrum (ZSS), which can be caused by mutations in 13 different PEX genes. As noted, ZSS usually affects multiple organs with disease onset at birth. Neurological symptoms include severe muscular hypotonia, peripheral neuropathy, seizures and failure to thrive.

Neuropathologically, neuronal migration defects and progressive symmetric white matter abnormalities in the central nervous system are characteristic features of the disease (Powers and Moser, 1998). There is currently no treatment available for ZSS and severely affected patients die within months.

Currently, five Pex gene knockout models have been generated in mice and recapitulate biochemical, clinical and pathological characteristics of the corresponding human phenotypes (Baes and Van Veldhoven, 2012). Tissue specific conditional knockouts have been extensively characterized for Pex5 and offer the opportunity to understand peroxisomal functions in individual cell types. Compared to complete Pex5 knockout mice (Baes et al., 1997), which die within days after birth and thus resemble patients with severe ZSS, oligodendrocyte specific Pex5 deficient mice (Cnp-Cre Pex5^{fl/fl}) are normal at birth (Kassmann et al., 2007). Cnp-Cre Pex5^{fl/fl} animals develop a slow but progressive impairment in motor coordination, which starts at month 3. By month six, many animals have moderate walking difficulties and almost none of the animals survive one year of age. Histopathologically, the disease is characterized by progressive demyelination and axonal loss, which becomes evident at the age of three months. In addition, a strong neuroinflammatory response, mainly composed of activated microglia cells, can be observed. Of note, microglia activation precedes demyelination and axonal damage in Cnp-Cre Pex5^{fl/fl} mice.

Quinoline-3-carboxamide derivatives such as laquinimod (5-chloro-N-ethyl-4-hydroxy-1-methyl-2-oxo-N-phenyl-1,2-dihydroquinoline-3-carboxamide) are useful in modulating innate immunity in animal models of MS and are currently evaluated for the treatment of multiple sclerosis.

Laquinimod is a novel synthetic compound with high oral bioavailability which has been suggested as an oral formulation for the treatment of Multiple Sclerosis (MS) (Polman, 2005; Sandberg-Wollheim, 2005). Laquinimod and its sodium salt form are described, for example, in U.S. Pat. No. 6,077,851. The mechanism of action of laquinimod is not fully understood.

Laquinimod showed a favorable safety and tolerability profile in multiple sclerosis (MS) patients in two phase III trials (Results of Phase III BRAVO Trial Reinforce Unique Profile of Laquinimod for Multiple Sclerosis Treatment; Teva Pharmaceuticals, Active Biotech Post Positive Laquinimod Phase 3 ALLEGRO Results).

Laquinimod has not been disclosed to be effective in treating leukodystrophies.

### BRIEF DESCRIPTION OF THE PRESENT INVENTION

In a first aspect, the present invention relates to compounds useful in the treatment of leukodystrophies as defined herein. In an embodiment, the compound is laquinimod, a quinolone derivative.

In a further aspect, the present invention relates to a method of treating leukodystrophies comprising the step of administering a compound as defined herein or a pharmaceutical composition containing the same to a subject in need thereof; in a preferred embodiment, the compound is laquinimod. In addition, the present invention relates to a method of treating hereditary central nervous system disorders comprising the step of administering a compound according to the present invention or a pharmaceutical composition containing the same to a subject in need thereof. Moreover, the present invention relates to a method for treating peroxisomal disorders comprising the step of administering a compound according to the present invention or a pharmaceutical composition containing the same to a subject in need thereof.

In particular, the invention provides a method of treating a subject suffering from leukodystrophy, the method comprising periodically administering to the subject an amount of laquinimod or pharmaceutically acceptable salt thereof effective to treat the subject.

This invention provides use of laquinimod in the manufacture of a medicament for treating a subject suffering from leukodystrophy.

This invention provides laquinimod for use in treating a subject suffering from leukodystrophy. This invention provides use of laquinimod in treating a subject suffering from leukodystrophy.

This invention provides a pharmaceutical composition comprising laquinimod for use in treating a subject suffering from leukodystrophy.

This invention provides a pharmaceutical oral unit dosage form of laquinimod for use in treating a subject suffering from leukodystrophy.

### BRIEF DESCRIPTION OF THE DRAWINGS

FIG. 1: FIG. 1 shows that laquinimod treatment improves the walking ability of Cnp-Cre Pex5^{fl/fl} Rag1^{-/-} mice. Cnp-Cre Pex5^{fl/fl} Rag1^{-/-} mice received either H₂O (Ctrl, n=18) or laquinimod (25 mg/kg, n=11) at 6 out 7 days/week by oral gavage. Treatment was initiated at the age of 6 weeks. Unpaired t test, **<0.01; ***<0.001.

FIG. 2: Quantification of axonal damage (APP), microglia/macrophage cell numbers (Mac3), demyelination and adult oligodendrocytes (NogoA) in the corpus callosum (CC) of CNP-Cre Pex^{fl/fl} RAG1^{-/-} mice at the age of 6 months.

FIG. 3: Titration of laquinimod to microglia cells stimulated with 1 μg/ml LPS and 5 mM ATP. Microglia cell supernatants were analyzed for IL1β by ELISA.

### DETAILED DESCRIPTION OF THE PRESENT INVENTION

The present invention relates in a first embodiment to a compound of the general formula (I)

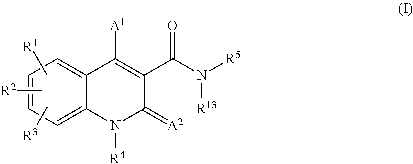

and the tautomers thereof wherein the groups A¹ and A² are interchanged and there is a 2,3-rather than a 3,4-double bond, for use in the treatment of leukodystrophy;
where R¹, R² and R³ are the same or different and are selected from the group consisting of: hydrogen; C₁-C₆ alkyl; C₁-C₆ alkenyl; C₁-C₅ alkoxy; C₁-C₆ alkylene; C₃-C₆ cycloalkyl; C₁-C₆ alkylthio; C₃-C₆ cycloakylthio; C₁-C₆ alkylsulfinyl; C₃-C₆ cycloalkylsulfinyl; aryl; acyl; heteroaryl; aralalkyl; allyl; carboxyl; amid; carbamoyl; carbonylamin; nitro; amino; cyano; trifluoromethyl; trifluoromethoxy; halogen; NO₂; OH; OCOR⁸; NR⁶R⁷; and NR⁶COR⁸; and where R¹ and R² or R² and R³ together may also be in the form of a methylenedioxy group; where R⁴ is selected from the group consisting of: C₁-C₆ alkyl; C₁-C₆ alkenyl; C₁-C₅ alkoxy; C₁-C₆ alkylene; C₃-C₆ cycloalkyl; C₁-C₆ alkylthio; C₃-C₆ cycloakylthio; C₁-C₆ alkylsulfinyl; C₃-C₆ cycloalkylsulfinyl; aryl; acyl; heteroaryl; aralalkyl; allyl; carboxyl; amid; carbamoyl; carbonylamin; nitro; amino; cyano; C₁-C₆ alkylene forming a ring with the 8-position carbon atom of the quinoline ring system; cycloalkyl; optionally mono- or disubstituted; preferred optionally mono- or disubstituted with substituents selected from the group consisting of C₁-C₆ alkyl, C₁-C₆ alkoxy, OH and OCOR⁸; and phenyl, optionally mono- or disubstituted, preferred optionally mono- or disubstituted with substituents selected from the group consisting of C₁-C₆ alkyl, C₁-C₆ alkoxy and halogen; and wherein R⁵ is selected from the group consisting of a five- or six-membered heterocyclic ring containing at most two heteroatoms selected from the group consisting of S and N, and being optionally mono- or disubstituted, preferred optionally mono- or disubstituted with substituents selected from the group consisting of C₁-C₆ alkyl, C₁-C₆ alkoxy, hydroxy and halogen; and wherein R⁵ may also be the group:

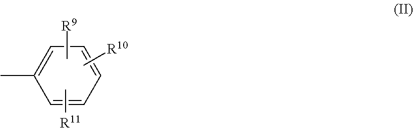

wherein R⁹, R¹⁰ and R¹¹ are the same or different and selected from the group consisting of: hydrogen; C₁-C₆ alkyl; C₁-C₆ alkenyl; C₁-C₅ alkoxy; C₁-C₆ alkylene; C₃-C₆ cycloalkyl; C₁-C₆ alkylthio; C₃-C₆ cycloakylthio; C₁-C₆ alkylsulfinyl; C₃-C₆ cycloalkylsulfinyl; aryl; acyl; heteroaryl; aralalkyl; allyl; carboxyl; amid; carbamoyl; carbonylamin; nitro; amino; trifluoromethyl; trifluoromethoxy; halogen; CN; SO₂CH₃; OH; OCOR⁶; NR⁶R⁷; NR⁶COR⁸; COOR¹²; OCH₂COOR¹²; CH₂COOR¹²; COR⁸; and

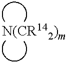

where each R¹⁴ are the same or different and are selected from the group consisting of: hydrogen; C₁-C₆ alkyl; C₁-C₆ alkenyl; C₁-C₅ alkoxy; C₁-C₆ alkylene; C₃-C₆ cycloalkyl; C₁-C₆ alkylthio; C₃-C₆ cycloakylthio; C₁-C₆ alkylsulfinyl; C₃-C₆ cycloalkylsulfinyl; aryl; acyl; heteroaryl; aralalkyl; allyl; carboxyl; amid; carbamoyl; carbonylamin; nitro; amino; cyano; trifluoromethyl; trifluoromethoxy; halogen; NO₂; OH; OCOR⁸; NR⁶R⁷; and NR⁶COR⁸ preferably wherein at least one of R¹⁴ are hydrogen;
wherein m is four or five; and where R⁹ and R¹⁰ or R¹⁰ and R¹¹ together also may be in the form of a methylenedioxy group;
wherein A¹ is selected from the group consisting of OR¹², OCOR⁸, NR⁶R⁷ and NR⁶COR⁸,
and wherein A² is selected from the group consisting of O and NR⁶; wherein R⁶, R⁷ and R⁸ are the same or different and selected from the group consisting of hydrogen, C₁-C₆ alkyl; C₁-C₆ alkenyl; C₁-C₅ alkoxy; C₁-C₆ alkylene; C₃-C₆ cycloalkyl; C₁-C₆ alkylthio; C₃-C₆ cycloakylthio; C₁-C₆ alkylsulfinyl; C₃-C₆ cycloalkylsulfinyl; aryl; acyl; heteroaryl; aralalkyl; allyl; carboxyl; amid; carbamoyl; carbonylamin; nitro; amino; cyano;
wherein R¹² is selected from the group consisting of C₁-C₆ alkyl and M; and
wherein M is selected from the group consisting of hydrogen and pharmaceutically acceptable inorganic and organic cations;
and wherein R¹³ is selected from the group consisting of hydrogen, C₁-C₆ alkyl, C₁-C₆ alkenyl, C₁-C₅ alkoxy, C₁-C₆ alkylene, C₃-C₆ cycloalkyl, C₁-C₆ alkylthio, C₃-C₆ cycloakylthio, C₁-C₆ alkylsulfinyl, C₃-C₆ cycloalkylsulfinyl, aryl, acyl, heteroaryl, aralalkyl, allyl, carboxyl, amid, carbamoyl, carbonylamin, nitro, amino, cyano, preferably C₁-C₆ alkyl, optionally substituted with a substituent selected from the group consisting of OH, OR⁸ and OCOR⁸, and C₁-C₆ alkenyl; provided that R¹³ is selected from the group consisting of C₁-C₆ alkyl, optionally substituted with a substituent selected from the group consisting of OH, OR⁸ and OCOR⁸, and C₁-C₆ alkenyl when R⁹, R¹⁰ and R¹¹ are selected from the group consisting of C₁-C₆ alkyl, C₁-C₆ alkenyl and C₁-C₆ alkoxy; and addition salts with pharmaceutically acceptable inorganic or organic acids.

In an embodiment of the present invention, the compound according the present invention for use in the treatment of Leukodystrophy is a compound wherein R¹³ is selected from the group consisting of C₁-C₆ alkyl optionally substituted, A¹ is OH and A² is O, R⁴ is C₁-C₃ alkyl and R⁵ is the group II as defined herein.

In a further embodiment, the compound is a compound for use in the treatment of leukodystrophy wherein the compound is a compound of general formula (III)

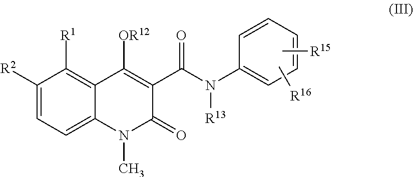

wherein
R¹³ is selected from methyl, ethyl, n-propyl, iso-propyl, n-butyl, iso-butyl, sec-butyl and allyl;
R¹² is selected from hydrogen and pharmaceutically acceptable inorganic and organic cations;
R¹ and R2 are the same or different and selected from hydrogen, methyl, ethyl, n-propyl, iso-propyl, n-butyl, iso-butyl, sec-butyl, methoxy, ethoxy, chloro, bromo, CF₃, and OCH_{X}F_{Y};
wherein
x=0-2,
y=1-3 with the proviso that
x+y=3;
or
R¹ and R² taken together are methylenedioxy;
R¹⁵ is hydrogen, a straight or branched, saturated or unsaturated C₁-C₆-alkyl or -alkenyl, a cyclic C₃-C₆-alkyl, a straight or branched C₁-C₆-alkoxy, a cyclic C₃-C₆-alkoxy, fluoro, chloro, bromo, trifluoromethoxy or trifluoromethyl; and
R¹⁶ is hydrogen, fluoro or chloro, with the proviso that R¹⁵ is fluoro or chloro only when R¹⁶ is fluoro or chloro
and any tautomer thereof.

Another embodiment relates to a compound being a compound of general formula (IV)

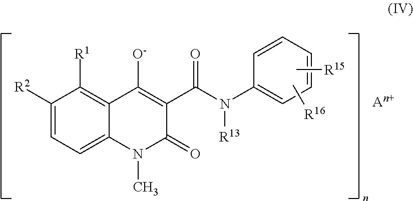

wherein
n is an integer of 1, 2 or 3;
Aⁿ⁺ is a mono- or multivalent metal cation selected from Li⁺, Na⁺, K⁺, Mg²⁺, Ca²⁺, Mn²⁺, Cu²⁺, Zn²⁺, Al³⁺ and Fe³⁺;
R¹³ is a straight or branched C₁-C₄-alkyl or -alkenyl or a cyclic C₃-C₄-alkyl;
R¹ and R² are the same or different and selected from hydrogen, straight or branched, saturated or unsaturated C₁-C₆-alkyl or -alkenyl, a cyclic C₃-C₆-alkyl, a straight or branched C₁-C₆-alkylthio, a cyclic C₃-C₆-alkylthio, a straight or branched C₁-C₆-alkylsulfinyl, a cyclic C₃-C₆-alkylsulfinyl, fluoro, chloro, bromo, trifluoromethyl or trifluoromethoxy; and
or
R¹ and R² taken together are methylenedioxy;
R¹⁵ is hydrogen, a straight or branched, saturated or unsaturated C₁-C₄-alkyl or -alkenyl, a cyclic C₃-C₄-alkyl, a straight or branched C₁-C₄-alkoxy, a cyclic C₃-C₄-alkoxy, fluoro, chloro, bromo or trifluoromethyl; and
R¹⁶ is hydrogen, fluoro or chloro, with the proviso that R¹⁶ is fluoro or chloro only when R¹⁵ is fluoro or chloro; optionally, an alkaline-reacting component maintaining the pH preferably above 8, or a salt with a divalent metal cation.

The compounds for use in treating leukodystrophy according to the present invention are e.g. selected from Roquinimex (4-hydroxy-N, 1-dimethyl-2-oxo-N-phenyl-1,2-dihydroquinoline-3-carboxamide) or laquinimod (5-chloro-N-ethyl-4-hydroxy-1-methyl-2-oxo-N-phenyl-1,2-dihydroquinoline-3-carboxamide).

In a further embodiment the compounds according to the present invention are for use in the treatment of hereditary central nervous systems disorders for example the compounds are for use in the treatment of peroxisomal disorders.

Another embodiment of the present invention refers to compounds according to the present invention for use in the treatment of Leukodystrophy selected from adrenoleukodystrophy, metachromatic Leukodystrophy, globoid cell leukodystrophy (Morbus Krabbe), Pelizaeus-Merzbacher disease, Canavan-Syndrom, vanishing white matter leukencephalopathy, Alexander disease, Refsum-Thiebaut disease, cerebrotendious xanthomatosis, Morbus Batten and Zellweger Syndrome.

In a particular embodiment the compounds according to the present invention are for use in the treatment of Zellweger Syndrome.

For example, the compounds according to the present invention are designed, prepared or adapted for oral administration.

Further, the present invention relates to a method of treating leukodystrophy comprising the step of administering a compound of formula (I) or any one of the compounds of formulae (III) or (IV) as defined herein or a pharmaceutical composition containing the same to a subject in need thereof.

In an embodiment, the method for treating leukodystrophy according to the present invention is a method of treating leukodystrophy selected from any one of adrenoleukodystrophy, metachromatic leukodystrophy, globoid cell leukodystrophy (Morbus Krabbe), Pelizaeus-Merzbacher disease, Canavan-Syndrom, vanishing white matter leukencephalopathy, Alexander disease, Refsum-Thiebaut disease, cerebrotendious xanthomatosis, Morbus Batten and Zellweger Syndrome.

In another embodiment, the present invention relates to a method of treating hereditary central nervous system disorders comprising the step of administering a compound of formula (I) or any one of the compounds of formulae (III) or (IV) as defined herein or a pharmaceutical composition containing the same to a subject in need thereof.

Moreover, the present invention relates to a method for treating peroxisomal disorders comprising the step of administering a compound of formula (I) or any one of the compounds of formulae (III) or (IV) as defined herein or a pharmaceutical composition containing the same to a subject in need thereof.

As used herein, and unless stated otherwise, each of the following terms shall have the definition set forth below.

As used herein, “laquinimod” means laquinimod acid or a pharmaceutically acceptable salt thereof, as well as derivatives as laquinimod such as deuterium enriched laquinimod, and salts thereof.

A “salt” is salt of the instant compounds which have been modified by making acid or base salts of the compounds. The term “pharmaceutically acceptable salt” in this respect, refers to the relatively non-toxic, inorganic and organic acid or base addition salts of compounds of the present invention.

As used herein, “treating” encompasses, e.g., inducing inhibition, regression, or stasis of the disorder. Specifically, treatment of a patient suffering from leukodystrophy includes, e.g., reducing a symptom of leukodystrophy in the subject, inducing clinical response, inhibiting disease progression, or inhibiting a disease complication in the subject.

“Inhibition” of disease progression or disease complication in a subject means preventing or reducing the disease progression and/or disease complication in the subject.

A “symptom” associated with leukodystrophy includes any clinical or laboratory manifestation associated with leukodystrophy and is not limited to what the subject can feel or observe.

As used herein, “a subject afflicted with leukodystrophy” means a subject who was been affirmatively diagnosed to have leukodystrophy.

As used herein, “leukodystrophy” includes all forms of leukodystrophy, including l8q Syndrome, Acute Disseminated Encephalomyeolitis (ADEM), Acute Disseminated Leukoencephalitis, Acute Hemorrhagic Leukoencephalopathy, Adrenoleukodystrophy X-Linked (ALD), Adrenomyeloneuropathy (AMN), Aicardi-Goutieres Syndrome, Alexander Disease, Adult-onset Autosomal Dominant Leukodystrophy (ADLD), Autosomal Dominant Diffuse Leukoencephalopathy (HDLS), Autosomal Dominant Late-Onset Leukoencephalopathy, Childhood Ataxia with diffuse CNS Hypomyelination (CACH or Vanishing White Matter Disease), Canavan Disease, Cerebral Autosomal Dominant Arteropathy with Subcortical Infarcts and Leukoencephalopathy (CADASIL), Cerebrotendinous Xanthomatosis (CTX), Craniometaphysical Dysplasia with Leukoencephalopathy, Cystic Leukoencephalopathy with RNASET2, Extensive Cerebral White Matter abnormality, Familial Adult-Onset Leukodystrophy, Familial Leukodystrophy, Globoid Cell Leukodystrophy (Krabbe Disease), Hereditary Adult Onset Leukodystrophy, Hypomyelination with Atrophy of the Basal Ganglia and Cerebellum (HABC), Hypomyelination, Hypogonadotropic, Hypogonadism and Hypodontia (4H Syndrome), Lipomembranous Osteodysplasia with Leukodystrophy (Nasu Disease), Metachromatic Leukodystrophy (MLD), Megalencephalic Leukodystrophy with subcortical Cysts (MLC), Neuroaxonal Leukoencephalopathy′ with axonal spheroids (Hereditary diffuse leukoencephalopathy with spheroids—HDLS), Oculodetatoldigital Dysplasia with cerebral white matter abnormalities, Orthochromatic Lleukodystrophy with pigmented glia, Ovarioleukodystrophy Syndrome, Pelizaeus Merzbacher Disease (X-linked spastic paraplegia), Refsum Disease, Sjogren-Larssen Syndrome, Sudanophilic Leukodystrophy, Van der Knaap Syndrome (Vacuolating Leukodystrophy with Subcortical Cysts or MLC), Vanishing White Matter Disease (VWM) or Childhood ataxia with diffuse central nervous system hypomyelination, (CACH), X-linked Adrenoleukodystrophy (X-ALD), Zellweger Syndrome (ZS), Neonatal Adrenoleukodystrophy (NALD), and Infantile Refsum Disease (IRD).

In the context of the present invention, the term “comprising”, “comprises”, “containing” or “contains” include the embodiments of “consisting of” or “consist”.

The term “halogen” as used herein includes the halogens F, Cl, Br and I, it is preferred that the halogen is CI or Br or I.

The term “C₁ to C₆” as used herein include compounds having C₁, C₂, C₃, C₄, C₅, C₆ carbon atoms. The term “C₁ to C₄” include C₁, C₂, C₃ or C₄ carbon atoms. The term “C₁ to C₃” include, C₁, C₂ or C₃ carbon atoms. The groups may be present in linear, branched or cyclic form.

The term “Alkyl” refers to a saturated aliphatic hydrocarbon including straight chain and branched chain groups. The alkyl group may be a C₁-C₆ alkyl group, like a C₁-C₄ alkyl group, e.g. C₁-C₃ alkyl group. “Alkyl” may be exemplified by groups such as methyl, ethyl, n-propyl, isopropyl, n-butyl, iso-butyl, sec-butyl and the like. Alkyl groups may be substituted or unsubstituted. Substituents may also be themselves substituted. When substituted, the substituent group is preferably, but not limited to, C₁-C₃ alkyl, aryl, amino, cyano, halogen, C₁-C₃ alkoxy or hydroxyl.

“Acyl” or “carbonyl” refers to the group —C(O)R wherein R is H, C₁-C₆ alkyl, like C₁-C₄ alkyl group, e.g. C₁-C₃ alkyl, aryl, heteroaryl, carbocyclic, heterocarbocyclic, C₁-C₆ alkyl aryl or C₁-C₆ alkyl heteroaryl. They may be substituted or unsubstituted.

“Alkoxy” refers to the group —O—R wherein R is acyl, alkyl, like C₁-C₈ alkyl, e.g. C₁-C₃ alkyl, aryl, carbocyclic, heterocarbocyclic, heteroaryl, C₁-C₆ alkyl aryl or C₁-C₆ alkyl heteroaryl. They may be substituted or unsubstituted.

“Aralkyl” refers to a radical in which an aryl group is substituted for a hydrogen atom of an alkyl group; e.g., C₆H₅CH₂—. They may be substituted or unsubstituted.

“Amino” refers to the group —NR′R″ wherein R′ and R″ are each, independently, hydrogen, alkyl, aryl, heteroaryl, C₁-C₃ alkyl aryl or C₁-C₃ alkyl heteroaryl. The R′ and R″ groups may themselves be linked to form a ring. They may be substituted or unsubstituted.

“Aryl” refers to an aromatic carbocyclic group. “Aryl” may be exemplified by phenyl or benzyl or naphthyl. The aryl group may be substituted or unsubstituted. Substituents may also be themselves substituted. When substituted, the substituent group is preferably, but not limited to, alkyl, alkoxy, heteroaryl, acyl, carboxyl, amido, carbamoyl, carbonylamino, nitro, amino, cyano, halogen or hydroxyl. The substituents may be positioned at various locations on an aryl group. For example, substituents on a phenyl group may be located at an ortho-position, a meta-position, the para-position, or combinations thereof.

The term “substituted” refers to C₁-C₆ alkyl, C₁-C₆ alkenyl, C₁-C₅ alkoxy, C₁-C₆ alkylene, C₃-C₆ cycloalkyl, C₁-C₆ alkylthio, C₃-C₆ cycloakylthio, C₁-C₆ alkylsulfinyl, C₃-C₆ cycloalkylsulfinyl, aryl, acyl, heteroaryl, aralalkyl, allyl, carboxyl, amid, carbamoyl, carbonylamin, nitro, amino, cyano, halogen or hydroxyl. Substituents may be positioned at various locations/positions of the same compound. Substituents may be substituted or unsubstituted.

Provided herein is a method of treating a subject suffering from leukodystrophy, the method comprising periodically administering to the subject an amount of laquinimod or pharmaceutically acceptable salt thereof effective to treat the subject.

In one embodiment, the pharmaceutically acceptable salt of laquinimod is laquinimod sodium.

In one embodiment, laquinimod is administered via oral administration.

In one embodiment, laquinimod is administered in a composition comprising the laquinimod and an amount of an amino acid.

In one embodiment, the amino acid is selected from lysine, glycine, proline, alanine, or histidine.

In one embodiment, the periodic administration is daily administration. In another embodiment, the periodic administration is more often than once daily. In another embodiment, the periodic administration is less often than once daily.

In one embodiment, the amount laquinimod administered is less than 0.6 mg/day. In another embodiment, the amount laquinimod administered is 0.1-40.0 mg/day. In another embodiment, the amount laquinimod administered is 0.1-2.5 mg/day. In another embodiment, the amount laquinimod administered is 0.25-2.0 mg/day. In another embodiment, the amount laquinimod administered is 0.5-1.2 mg/day. In another embodiment, the amount laquinimod administered is 0.25 mg/day. In another embodiment, the amount laquinimod administered is 0.3 mg/day. In another embodiment, the amount laquinimod administered is 0.5 mg/day. In another embodiment, the amount laquinimod administered is 0.6 mg/day. In another embodiment, the amount laquinimod administered is 1.0 mg/day. In another embodiment, the amount laquinimod administered is 1.2 mg/day. In another embodiment, the amount laquinimod administered is 1.5 mg/day. In yet another embodiment, the amount laquinimod administered is 2.0 mg/day.

In one embodiment, the periodic administration of laquinimod continues for at least 3 days. In another embodiment, the periodic administration of laquinimod continues for more than 30 days. In another embodiment, the periodic administration of laquinimod continues for more than 42 days. In another embodiment, the periodic administration of laquinimod continues for 8 weeks or more. In another embodiment, the periodic administration of laquinimod continues for at least 12 weeks. In another embodiment, the periodic administration of laquinimod continues for at least 24 weeks. In another embodiment, the periodic administration of laquinimod continues for more than 24 weeks. In another embodiment, the periodic administration of laquinimod continues for 6 months or more.

In one embodiment, the subject is a human.

In one embodiment, the leukodystrophy is 18q Syndrome, Acute Disseminated Encephalomyeolitis (ADEM), Acute Disseminated Leukoencephalitis, Acute Hemorrhagic Leukoencephalopathy, Adrenoleukodystrophy X-Linked (ALD), Adrenomyeloneuropathy (AMN). Aicardi-Goutieres Syndrome, Alexander Disease, Adult-onset Autosomal Dominant Leukodystrophy (ADLD), Autosomal Dominant Diffuse Leukoencephalopathy (HDLS), Autosomal Dominant Late-Onset Leukoencephalopathy, Childhood Ataxia with diffuse CNS Hypomyelination (CACI-I or Vanishing White Matter Disease), Canavan Disease, Cerebral Autosomal Dominant Arteropathy with Subcortical Infarcts and Leukoencephalopathy (CADASIL), Cerebrotendinous Xanthomatosis (CTX), Craniometaphysical Dysplasia with Leukoencephalopathy, Cystic Leukoencephalopathy with RNASET2, Extensive Cerebral White Matter abnormality, Familial Adult-Onset Leukodystrophy, Familial Leukodystrophy, Globoid Cell Leukodystrophy (Krabbe Disease), Hereditary Adult Onset Leukodystrophy, Hypomyelination with Atrophy of the Basal Ganglia and Cerebellum (HABC), Hypomyelination, Hypogonadotropic, Hypogonadism and Hypodontia (4H Syndrome), Lipomembranous Osteodysplasia with Leukodystrophy (Nasu Disease), Metachromatic Leukodystrophy (MLD), Megalencephalic Leukodystrophy with subcortical Cysts (MLC), Neuroaxonal Leukoencephalopathy with axonal spheroids (Hereditary diffuse leukoencephalopathy with spheroids—HDLS), Oculodetatoldigital Dysplasia with cerebral white matter abnormalities, Orthochromatic Lleukodystrophy with pigmented glia, Ovarioleukodystrophy Syndrome, Pelizaeus Merzbacher Disease (X-linked spastic paraplegia), Refsum Disease, Sjogren-Larssen Syndrome, Sudanophilic Leukodystrophy, Van der Knaap Syndrome (Vacuolating Leukodystrophy with Subcortical Cysts or MLC), Vanishing White Matter Disease (VWM) or Childhood ataxia with diffuse central nervous system hypomyelination, (CACH), X-linked. Adrenoleukodystrophy (X-ALD), Zellweger Syndrome (ZS), Neonatal Adrenoleukodystrophy (NALD), or Infantile Refsum Disease (IRD).

In one embodiment, the leukodystrophy is a peroxisome biogenesis disorder.

In one embodiment, the peroxisome biogenesis disorder is Zellweger Syndrome, Neonatal Adrenoleukodystrophy, or Infantile Refsum Disease.

In one embodiment, the peroxisome biogenesis disorder is Zellweger Syndrome.

In one embodiment, the amount of laquinimod is effective to reduce a symptom of leukodystrophy in the subject comparing to that in a subject afflicted with leukodystrophy not treated with laquinimod.

In one embodiment, the symptom of leukodystrophy is motor dysfunction, delayed development of motor skills, plateau in development of motor skills, regression in motor skills, rigidity, dystonia, ataxia, or bulbar symptoms.

In one embodiment, the symptom of leukodystrophy is cognitive impairment, developmental delay, intellectual disability, or dementia.

In one embodiment, the symptom of leukodystrophy is nystagmus, irritability, titubation, autonomic dysfunction, encephalopathy, endocrine dysfunction, ophthalmologic abnormalities, cortical visual impairment, dental abnormalities, dysmorphic physical features, tendinous xanthomas, skeletal abnormalities, bony abnormalities, hearing impairment, hepatosplenomegaly, cutaneous abnormalities, ovarian dysgenesis or gastrointestinal symptoms.

In one embodiment, the symptom of leukodystrophy is white matter abnormality. In one embodiment the white matter abnormality is demyelination, dysmyelination, or hypomyelination. In one embodiment, the symptom is axonal loss.

In one embodiment, the amount of laquinimod is effective to reduce a symptom of peroxisome biogenesis disorder (PBD) in the subject comparing to that in a subject afflicted with PBD not treated with laquinimod.

In one embodiment, the symptom of PBD is craniofacial abnormalities, high forehead, hypoplastic supraorbital ridges, epicanthal folds, midface hypoplasia, and large anterior fontanel, eye abnormalities, corneal clouding, cataracts, flaucoma, optic atrophy, retinal anomalies, neuronal migration defects, polymicrogyria, Purkinje cell heterotopia, olivary nucleus abnormities, hepatomegaly, renal cysts, chondrodysplasia punctate, hypotonia, seizures, inability to feed, impaired neonatal and deep tendon reflexes, impaired spontaneous movement, spasticity, sensorineural hearing loss, retinitis pigmentosa, disturbed ossification, shortening of the proximal long bones with metaphyseal cupping, coronal clefts of the vertebral bodies, epiphyseal stippling, contractures, bilateral cataracts, abnormal faces with frontal bossing, depressed nasal bridge, small nose, ichthyosis, central nervous system abnormalities, cerebral and cerebellar atrophy, abnormalities of myelination, neuronal migration defects, growth retardation, psychomotor retardation, respiratory complications, congenital cataracts, chondrodysplasia, rhizomelia, mental deficiency, growth deficiency, or learning disability.

In one embodiment, the amount of laquinimod is effective to reduce a symptom of Zellweger syndrome (ZS) in the subject comparing to that in a subject afflicted with ZS not treated with laquinimod.

In one embodiment, the symptom of ZS is craniofacial abnormalities, high forehead, hypoplastic supraorbital ridges, epicanthal folds, midface hypoplasia, and large anterior fontanel, eye abnormalities, corneal clouding, cataracts, flaucoma, optic atrophy, retinal anomalies, neuronal migration defects, polymicrogyria, Purkinje cell heterotopia, olivary nucleus abnormities, hepatomegaly, renal cysts, chondrodysplasia punctate, hypotonia, seizures, inability to feed, impaired neonatal and deep tendon reflexes, or impaired spontaneous movement.

In one embodiment, the amount of laquinimod is effective to reduce a symptom of Neonatal Adrenoleukodystrophy (NALD) in the subject comparing to that in a subject afflicted with NALD not treated with laquinimod.

In one embodiment, the symptom of NALD is craniofacial abnormalities, high forehead, hypoplastic supraorbital ridges, epicanthal folds, midface hypoplasia, and large anterior fontanel, eye abnormalities, corneal clouding, cataracts, flaucoma, optic atrophy, retinal anomalies, neuronal migration defects, polymicrogyria, Purkinje cell heterotopia, olivary nucleus abnormities, hepatomegaly, renal cysts, chondrodysplasia punctate, hypotonia, seizures, inability to feed, impaired neonatal and deep tendon reflexes, impaired spontaneous movement, spasticity, sensorineural hearing loss, or retinitis pigmentosa.

In one embodiment, the amount of laquinimod is effective to reduce a symptom of Infantile Refsum Disease (IRD) in the subject comparing to that in a subject afflicted with IRD not treated with laquinimod.

In one embodiment, the symptom of IRD is craniofacial abnormalities, high forehead, hypoplastic supraorbital ridges, epicanthal folds, midface hypoplasia, and large anterior fontanel, eye abnormalities, corneal clouding, cataracts, flaucoma, optic atrophy, retinal anomalies, neuronal migration defects, polymicrogyria, Purkinje cell heterotopia, olivary nucleus abnormities, hepatomegaly, renal cysts, chondrodysplasia punctate, hypotonia, seizures, inability to feed, impaired neonatal and deep tendon reflexes, impaired spontaneous movement, spasticity, sensorineural hearing loss, or retinitis pigmentosa.

In one embodiment, the symptom is reduced by at least 10%. In another embodiment, the symptom is reduced by at least 20%. In another embodiment, the symptom is reduced by at least 30%. In another embodiment, the symptom is reduced by at least 50%. In another embodiment, the symptom is reduced by at least 70%. In another embodiment, the symptom is reduced by more than 100%. In another embodiment, the symptom is reduced by more than 300%. In another embodiment, the symptom is reduced by more than 1000%.

In one embodiment, laquinimod is administered as add-on therapy to or in combination with one or more other treatment for leukodystrophy.

In one embodiment, the other treatment for leukodystrophy is chenodeoxycholic acid, clofarabine, melphalan, alemtuzumab, mycophenolate mofetil, cyclosporine A, hydroxyurea, rabbit antithymocyte globulin, fludarabine, busulfan, cyclophosphamide, methylprednisolone, granulocyte colony-stimulating factor, granulocyte-macrophage colony-stimulating factor, N-acetylcysteine, celecoxib, vitamin E, alpha lipoic acid, campath-1H, cyclophosphamide, Lorenzo's oil, sobetirome, filgrastim, triheptanoim, glyceryl triacetate (GTA), chenodeoxycholic acid, lovastatin, betaine, and/or nutropin AQ.

This invention provides use of laquinimod in the manufacture of a medicament for treating a subject suffering from leukodystrophy.

This invention provides laquinimod for use in treating a subject suffering from leukodystrophy.

This invention provides use of laquinimod in treating a subject suffering from leukodystrophy.

This invention provides a pharmaceutical composition comprising laquinimod for use in treating a subject suffering from leukodystrophy.

This invention provides a pharmaceutical oral unit dosage form of laquinimod for use in treating a subject suffering from leukodystrophy.

The compounds according to the present invention may be used in form of its free compounds or of salts thereof or in form of solvates, like hydrates. For example, the compounds according to the present invention may be administered in form of pharmaceutically acceptable salts thereof.

As used herein, the term “pharmaceutically acceptable salts thereof”, refers to salts which are non-toxic when administered to human or animals. Salts useful according to the present invention include hydrochlorides, hydrobromides, hydroiodides, sulfates, bisulfates, nitrates, citrates, tatrates, bitatrates, phosphates, hydrogenphosphates, dihydrogenphosphates, carbonates, hydrogencarbonates, malates, maleates, fumarates, succinates, acetates, terephthalates, laurates, palmitates, pamoates, pectinates, besilates, ciclotates, closilates, esilates, gluconates, hyclates, isethionates, lactobionates, mesylates, orotates, tosylates, xinafoates as well as salts with sodium, potassium, calcium, magnesium, deanol, diolamine, edamine, epolamine, erbumine, meglumine, olamine, trometamol.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated. by reference into this application.

The route of administration of the compounds of the present invention depends on the formulation in use. That is, the compounds according to the present invention may be administered in form of infusion, in form of capsules or other suitable forms, like tablets.

As mentioned, administration may depend on the form of the pharmaceutical composition used. For example, the pharmaceutical composition may be in solid form or fluid form for enteral or parenteral application.

In a further embodiment, the present invention relates to a pharmaceutical composition comprising one or more compounds according to the present invention.

Preferably, the pharmaceutical composition comprising the compounds according to the present invention is intended for the treatment of humans and/or animals.

The skilled person is well aware of suitable diluents, excipients, or carriers.

The pharmaceutical composition may be administered with a physiologically acceptable carrier to a patient, as described herein. In a specific embodiment, the term “pharmaceutically acceptable” means approved by a regulatory agency or other generally recognized pharmacopoeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatine, malt, rice, flour, chalk, silica gel, sodium stearate, glycerol monostearate, talc, sodium chloride, phosphates, hydrogenphosphates, dihydrogenphosphates, dried skim milk, glycerol, propyleneglycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, patches and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium, carbonate, etc. Examples of suitable pharmaceutical carriers are described in “Remington's Pharmaceutical Sciences” by E. W. Martin (18th ed., Mack Publishing Co., Easton, Pa. (1990)). Such compositions will contain a therapeutically effective amount of the aforementioned compounds according to the present invention, preferably in purified form, together with a suitable amount of carrier so as to provide the form for proper administration to the patient. The formulation should suit the mode of administration.

Typically, pharmaceutically or therapeutically acceptable carrier is a carrier medium which does not interfere with the effectiveness of the biological activity of the active ingredients and which is not toxic to the host or patient.

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

As used herein, “about” in the context of a numerical value or range means±10% of the numerical value or range recited or claimed.

“Administration” means the giving of, dispensing of, or application of medicines, drugs, or remedies to a subject to relieve or cure a pathological condition. Oral administration is one way of administering the instant compounds to the subject.

In another preferred embodiment, the composition is formulated in accordance with routine procedures as a pharmaceutical composition adapted for intravenous or oral administration to human beings. Typically, compositions for intravenous administration are solutions in sterile isotonic aqueous buffer. Where necessary, the composition may also include a solubilizing agent and a local anaesthetic such as lidocaine to ease pain at the site of the injection. Generally, the ingredients are supplied either separately or mixed together in a unit dosage form, for example, as a dry lyophilised powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The pharmaceutical composition for use in connection with the invention can be formulated as neutral or salt forms. Pharmaceutically acceptable salts include those formed with anions such as those derived from hydrochloric, phosphoric, acetic, oxalic, tartaric acid, etc., and those formed with cations such as those derived from sodium, potassium, ammonium, calcium, ferric hydroxides, isopropylamine, triethylamine, 2-ethylamino ethanol, histidine, procaine, etc.

“Therapeutically- or pharmaceutically-effective amount” as applied to the compositions of the instant invention refers to the amount of composition sufficient to induce a desired biological result. That result can be alleviation of the signs, symptoms, or causes of a disease, or any other desired alteration of a biological system.

In vitro assays may optionally be employed to help identifying optimal dosage ranges. The precise dose to be employed in the formulation will also depend on the route of administration, and the seriousness of the disease or disorder, and should be decided according to the judgement of the practitioner and each patient's circumstances. Effective doses may be extrapolated from dose-response curves derived from in vitro or animal model test systems. Preferably, the pharmaceutical composition is administered directly or in combination with an adjuvant. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

In the context of the present invention the term “subject” means an individual in need of a therapy that can be alleviated or cured by administering the compounds according to the present invention to the individual. Preferably, the subject is a vertebrate, even more preferred a mammal, particularly preferred a human.

The term “administered” means administration of a therapeutically effective dose of the aforementioned pharmaceutical composition comprising the compounds according to the present invention.

The methods are applicable to both human therapy and veterinary applications. The compounds described herein having the desired therapeutic activity may be administered in a physiologically acceptable carrier to a patient, as described herein. Depending upon the manner of introduction, the compounds may be formulated in a variety of ways as discussed below. The concentration of therapeutically active compound in the formulation may vary from about 0.1-100 wt %. The agents may be administered alone or in combination with other treatments.

As used herein, an “amount” or “dose” of laquinimod as measured in milligrams refers to the milligrams of laquinimod acid present in a preparation, regardless of the form of the preparation. Therefore, a “dose of 0.5 mg laquinimod” means the amount of laquinimod acid in a preparation is 0.5 mg, regardless of the form of the preparation. Similarly, a “dose of 1 mg laquinimod” means the amount of laquinimod acid in a preparation is 1 mg, regardless of the form of the preparation. Thus, when in the form of a salt, e.g. a laquinimod sodium salt, the weight of the salt form necessary to provide a dose of 0.5 mg laquinimod would be greater than 0.5 mg due to the presence of the additional salt ion.

As used herein, “combination” means an assemblage of reagents for use in therapy either by simultaneous or contemporaneous administration. Simultaneous administration refers to administration of an admixture (whether a true mixture, a suspension, an emulsion or other physical combination) of the reagents. In this case, the combination may be the admixture or separate containers of the reagents that are combined just prior to administration. Contemporaneous administration refers to the separate administration of the reagents at the same time, or at times sufficiently close together that a synergistic activity or an activity that is additive or more than additive relative to the activity of either reagents alone is observed.

As used herein, “effective” when referring to an amount of laquinimod refers to the quantity of a laquinimod that is sufficient to yield a desired therapeutic response without undue adverse side effects (such as toxicity, irritation, or allergic response) commensurate with a reasonable benefit/risk ratio when used in the manner of this invention.

It is understood that where a parameter range is provided, all integers within that range, and tenths and hundreds thereof, are also provided by the invention. For example, “0.2-2.0 mg/day” includes 0.2 mg/day, 0.25 mg/day, 0.3 mg/day, 0.4 mg/day, 0.5 mg/day, 0.6 mg/day etc. up to 2.0 mg/day.

A pharmaceutically acceptable salt of laquinimod. as used in this application includes lithium, sodium, potassium, magnesium, calcium, manganese, copper, zinc, aluminum and iron. Salt formulations of laquinimod and the process for preparing the same are described, e.g., in U.S. Patent Application Publication No. US 2005/0192315 and PCT International Application Publication No. WO 2005/074899, which are hereby incorporated by reference into this application.

A dosage unit may comprise a single compound or mixtures of compounds thereof. A dosage unit can be prepared for oral dosage forms, such as tablets, capsules, pills, powders, and granules.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit is preferably in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined. with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like.

Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Patent Application Publication No. US 2005/0192315, PCT International Application Publication Nos. WO 2005/074899, WO 2007/047863, and WO 2007/146248.

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

This invention will be better understood by reference to the Experimental Details which follow, but those skilled in the art will readily appreciate that the specific experiments detailed are only illustrative of the invention as described more fully in the claims which follow thereafter.

The administration of the pharmaceutical composition can be done in a variety of ways as discussed above, including, but not limited to, orally, subcutaneously, intravenously, intra-arterially, intranodally, intramedullarily, intrathecally, intraventricularly, intranasally, intrabronchially, transdermally, intrarectally, intraperitoneally, intramuscularly, intrapulmonarily, vaginally, rectally, or intraocularly. In some instances, for example, in the treatment of wounds and inflammation, the pharmaceutically effective agent may be directly applied as a solution dry spray.

The attending physician and clinical factors will determine the dosage regimen. A typical dose can be, for example, in the range of 0.0001 to 2000 mg, preferably about 0.1 to 1.5 mg; however, doses below or above this exemplary range are envisioned, especially considering the aforementioned factors.

The administration and the method for the treatment according to the present invention may be effected by any route of administration including oral, parenteral, such as subcutaneous, intravenous, intramuscular, intraperitoneal, intrathecal, transdermal, transmucosal, subdural, nasal, local or topical via iontophoresis, sublingual, by inhalation spray, aerosol or rectally and the like in dosage units formulations optionally comprising conventional pharmaceutically acceptable excipients, diluents or carriers.

The present invention will be described by way of examples without limiting the same.

### EXAMPLES

### Materials and Methods

Knockout mice of one or more of the following strains are used: Cnp-Pex5, Cnp1-Cre*PEX5flox/flox, Pex5^{flox/flox*}Cnp-Cre/+, Nestin-Pex5, NEX-Pex5, and GFAP-Pex5^{-/-}.

Knockout mice exhibits neurological problems including motoric and coordination deficits and cognitive impairment. Mutant mice exhibited behavioral abnormalities including hindlimb ataxia, kyphosis, hindlimb paresis, forelimb ataxia, tremor, hindlimb paralysis, and passiveness (Kassmann et al., 2007; Bottelbergs et al., 2010; Kassmann et al., 2011). Knockout mice also exhibits accumulation of lipid droplets, accumulation of very long-chain fatty acids (VLCFA), depletion of plasmalogens, impairment in the formation and maintenance of myelin, axonal degeneration, astrogliosis, and microgliosis (Kassmann et al., 2007; Bottelbergs et al., 2010; Kassmann et al., 2011). The effect of various doses of laquinimod on knockout mice is assessed.

### Results:

Administration of laquinimod improves motor function in knockout mice.

Administration of laquinimod reduces hindlimb ataxia, kyphosis, hindlimb paresis, forelimb ataxia, tremor, hindlimb paralysis, and/or passiveness in mice.

Administration of laquinimod improves cognitive function in mice.

Administration of laquinimod reduces accumulation of lipid droplets, accumulation of very long-chain fatty acids (VLCFA), depletion of plasmalogens, impairment in the formation andmaintenance of myelin, axonal degeneration, astrogliosis, and/or microgliosis in mice.

### Example 1

### Mouse Strains.

Cnp-Cre Pex5^{fl/fl} Rag1^{-/-} mice were generated by crossbreeding of Cnp-Cre Pex5^{fl/fl} mice with Rag1^{-/-} mice. Mice of both sexes received 25 mg/kg Laquinimod or water by oral gavage 6 out 7 days/week and treatment was initiated at the age of 6 weeks.

### Behavioral Analyses.

Motor coordination was assessed with a balanced beam test. Mice were put on a beam (width 1.5 cm) and allowed to run toward a hiding box. After a training period, the time to pass a distance of 0.6 m was measured (three repeats per time point).

### Histological and Morphological Analyses.

Mice were perfused transcardially at month 6 with cold PBS followed by 4% paraformaldehyde (PFA). Brains and spinal cords were post-fixed for 2 days and then paraffin-embedded. Sections between 0.5-1 μm were cut and processed for immunohistochemistry (IHC) according to standard protocols. Demyelination was evaluated on sections stained with luxol fast blue (LFB). For the staining of macrophages the antibody MAC387 (mouse anti-L1 antibody MAC387, GeneTex, 1:150) was used. Axonal density was evaluated on Bielschowsky silver stained sections.

### Results

Laquinimod treatment significantly improves the walking ability of Cnp-Cre Pex5^{fl/fl} Rag1^{-/-} mice

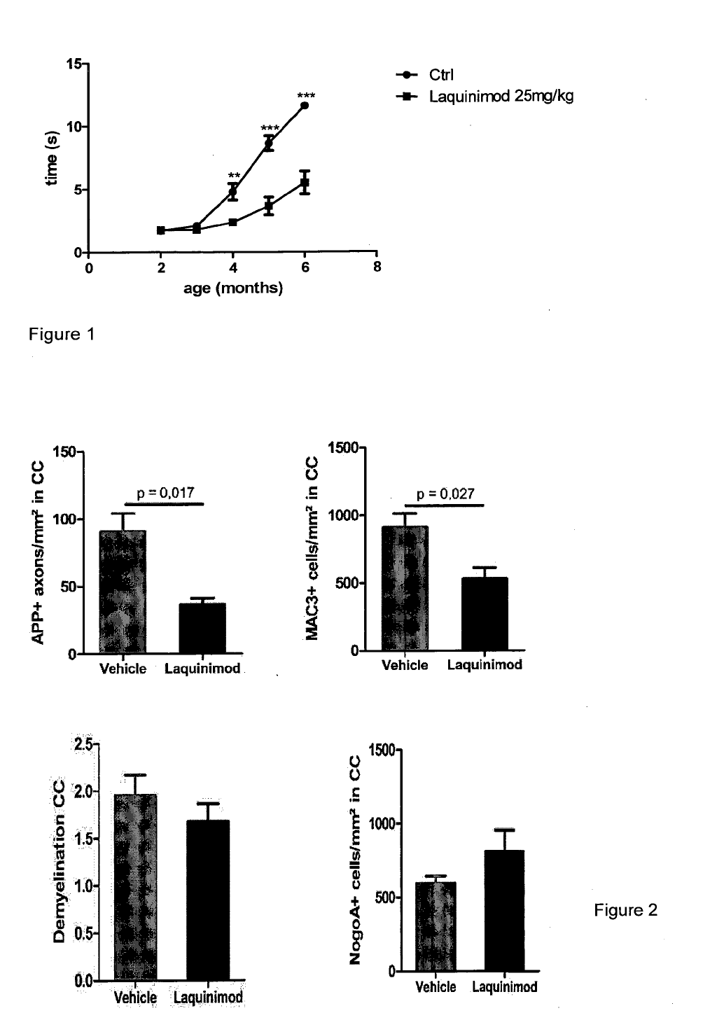

In order to improve the progressive walking impairment of Cnp-Cre Pex5^{fl/fl} Rag1^{-/-} mice, animals were treated with laquinimod or water at the age of 6 weeks up to month 6. As shown in FIG. 1, laquinimod treated mice started to perform significantly better on an elevated beam test than H₂O treated control mice at the age of 4 months. The highly significant clinical improvement in motor coordination was maintained throughout the experiment.

Laquinimod reduces microglia activation, demyelination and axonal loss in Cnp-Cre Pex5^{fl/fl} Rag1^{-/-} mice.

Treated and control mice were analyzed for microglia activation, demyelination and axonal loss to identify the pathological substrate of the impressive clinical benefit. It had been found that laquinimod treatment reduced the widespread microglia activation seen in water treated controls. Furthermore myelin loss in the corpus callosum was less extensive and axons were better preserved, which offer a rational explanation for the observed therapeutic benefits.

That is, in order to identify the mechanisms how laquinimod is beneficial in the CNP-Cre Pex^{fl/fl} RAG1^{-/-} mouse model, we analyzed and quantified the extent of demyelination, axonal damage and macrophage and adult oligodendrocyte numbers in immunohistochemically stained sections of the corpus callosum at month 6 in laquinimod and vehicle treated mice. Laquinimod treatment significantly reduced the amount of axonal damage and the microglia/macrophage cell numbers in the corpus callosum at the age of 6 months. The extent of demyelination was diminished and the numbers of preserved adult oligodendrocytes were increased, see FIG. 2. From the left to right, FIG. 2 shows reduced numbers of APP positive axons in laquinimod treated mice; reduced number of MAC positive cells (macrophage) in laquinimod-treated mice; reduced amount of demyelination in the CC of laquinimod treated mice; and a higher number of Nogo positive cells in lalaquinimod treated mice.

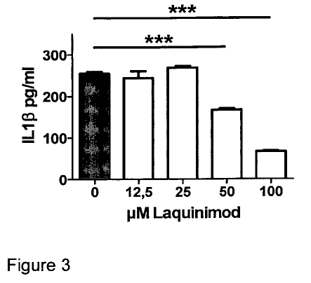

Next, whether laquinimod directly influences the neurotoxicity of microglia cells was analyzed Data show that laquinimod significantly reduced the amount of IL1β in LPS stimulated microglia cell cultures at high concentrations, see FIG. 3.

To conclude, Cnp-Cre Pex5^{fl/fl} Rag1^{-/-} mice treated with laquinimod maintain their walking ability significantly better than water treated controls. In addition, microglia activation, demyelination and axonal loss were significantly reduced in treated mice compared to controls. In summary, we provide evidence for meaningful neuropathological correlates for the clinical benefit observed in laquinimod treated CNP-Cre Pex^{fl/fl} RAG1^{-/-} mice. Furthermore, laquinimod is capable of reducing the amount of microglia-secreted neurotoxic cytokines.

### DISCUSSION

Cnp-Cre Pex5^{fl/fl} mice are a suitable model for a poorly understood neurodegenerative disease process, which affects the CNS white matter in patients with ZSS. Pathogenic factors, which have been implicated in this neurodegenerative disease process, include the loss of peroxisomal products such as plasmalogens, the accumulation of peroxisomal substrates such as very long chain fatty acids and more recently mitochondrial dysfunction, oxidative stress and an innate driven inflammatory response. As demonstrated herein, demyelination and axon loss can be reduced by Laquinimod argue in favor of a relevant contribution of the innate driven inflammatory response for white matter and axonal neurodegeneration in this disorder. In line with it, ramified, activated microglia cells were less abundant in treated mice compared to controls. Neuroinflammatory glial responses may contribute to neurodegeneration seen in parkinson's disease, alzheimer's disease and in amyotrophic lateral sclerosis through the excessive production of inflammatory cytokines, proteases and free radicals. Evidence has recently provided that laquinimod inhibits microglia and astrocyte activation and thus reduces demyelination and axonal injury in the cuprizone model (Bruck et al., 2012) and EAE model (Mishra et al., 2014) respectively.

### Effect of Laquinimod for Leukodystrophies in Humans

A trial is conducted to evaluate the safety, tolerability and clinical effect of laquinimod in leukodystrophy human patients.

Study Population and Number of Subjects

Patients with leukodystrophy are enrolled.

### Inclusion Criteria:

The patient has been affirmatively diagnosed to have leukodystrophy. The patient exhibits one or more of the following symptoms: motor dysfunction, delayed development of motor skills, plateau in development of motor skills, regression in motor skills, rigidity, dystonia, ataxia, bulbar symptoms, cognitive impairment, developmental delay, intellectual disability, dementia, nystagmus, irritability, titubation, autonomic dysfunction, encephalopathy, endocrine dysfunction, ophthalmologic abnormalities, cortical visual impairment, dental abnormalities, dysmorphic physical features tendinous xanthomas, skeletal abnormalities, bony abnormalities, hearing impairment, hepatosplenomegaly, cutaneous abnormalities, ovarian dysgenesis or gastrointestinal symptoms.

### Example 2: Investigational Medicinal Product and Dosage

### Laquinimod/Matching Placebo

Capsule(s) containing laquinimod and/or matching placebo are administered orally once daily at dosages approved by the study Safety Committee.

### Study Design

Patients are randomized into one of the treatment arms in a ratio that allows for reaching an overall target enrollment.

### Results

The administration of laquinimod alleviates or eliminates one or more symptoms of leukodystrophy. The administration of laquinimod alleviates or eliminates motor symptoms of leukodystrophy.

### Example 3: Effect of Laquinimod for Peroxisome Biogenesis Disorder (PBD) in Humans

A trial is conducted to evaluate the safety, tolerability and clinical effect of laquinimod for PBD in human patients.

### Study Population and Number of Subjects

Patients with PBD are enrolled.

### Inclusion Criteria:

The patient has been affirmatively diagnosed to have PBD. The patient exhibits one or more of the following symptoms: craniofacial abnormalities, high forehead, hypoplastic supraorbital ridges, epicanthal folds, midface hypoplasia, and large anterior fontanel, eye abnormalities, corneal clouding, cataracts, flaucoma, optic atrophy, retinal anomalies, neuronal migration defects, polymicrogyria, Purkinje cell heterotopia, olivary nucleus abnormities, hepatomegaly, renal cysts, chondrodysplasia punctate, hypotonia, seizures, inability to feed, impaired neonatal and deep tendon reflexes, impaired spontaneous movement, spasticity, sensorineural hearing loss, retinitis pigmentosa, disturbed ossification, shortening of the proximal long bones with metaphyseal cupping, coronal clefts of the vertebral bodies, epiphyseal stippling, contractures, bilateral cataracts, abnormal faces with frontal bossing, depressed nasal bridge, small nose, ichthyosis, central nervous system abnormalities, cerebral and cerebellar atrophy, abnormalities of myelination, neuronal migration defects, growth retardation, psychomotor retardation, respiratory complications, congenital cataracts, chondrodysplasia, rhizomelia, mental deficiency, growth deficiency, and learning disability.

### Investigational Medicinal Product and Dosage

### Laquinimod/Matching Placebo

Capsule(s) containing laquinimod and/or matching placebo are administered orally once daily at dosages approved by the study Safety Committee.

### Study Design

Patients are randomized into one of the treatment arms in a ratio that allows for reaching an overall target enrollment.

### Results

The administration of laquinimod alleviates or eliminates one or more symptoms of PBD. The administration of laquinimod alleviates or eliminates motor symptoms of PBD.

### Example 3.1: Effects of Laquinimod for Zellweger Syndrome (ZS) in Humans

The administration of laquinimod alleviates or eliminates one or more of the following symptoms of ZS: craniofacial abnormalities, high forehead, hypoplastic supraorbital ridges, epicanthal folds, midface hypoplasia, and large anterior fontanel, eye abnormalities, corneal clouding, cataracts, flaucoma, optic atrophy, retinal anomalies, neuronal migration defects, polymicrogyria, Purkinje cell heterotopia, olivary nucleus abnormities, hepatomegaly, renal cysts, chondrodysplasia punctate, hypotonia, seizures, inability to feed, impaired neonatal and deep tendon reflexes, and impaired spontaneous movement.

### Example 3.2: Effects of Laquinimod for Neonatal Adrenoleukodystrophy (NALD) in Humans

The administration of laquinimod alleviates or eliminates one or more of the following symptoms of NALD: craniofacial abnormalities, high forehead, hypoplastic supraorbital ridges, epicanthal folds, midface hypoplasia, and large anterior fontanel, eye abnormalities, corneal clouding, cataracts, flaucoma, optic atrophy, retinal anomalies, neuronal migration defects, polymicrogyria, Purkinje cell heterotopia, olivary nucleus abnormities, hepatomegaly, renal cysts, chondrodysplasia punctate, hypotonia, seizures, inability to feed, impaired neonatal and deep tendon reflexes, impaired spontaneous movement, spasticity, sensorineural hearing loss, and retinitis pigmentosa.

### Example 3.3: Effects of Laquinimod for Infantile Refsum Disease (IRD) in Humans

The administration of laquinimod alleviates or eliminates one or more of the following symptoms of IRD: craniofacial abnormalities, high forehead, hypoplastic supraorbital ridges, epicanthal folds, midface hypoplasia, and large anterior fontanel, eye abnormalities, corneal clouding, cataracts, flaucoma, optic atrophy, retinal anomalies, neuronal migration defects, polymicrogyria, Purkinje cell heterotopia, olivary nucleus abnormities, hepatomegaly, renal cysts, chondrodysplasia punctate, hypotonia, seizures, inability to feed, impaired neonatal and deep tendon reflexes, impaired spontaneous movement, spasticity, sensorineural hearing loss, and retinitis pigmentosa.

In summary, clinical and histopathological evidence is provided for a beneficial effect of laquinimod in a mouse model of ZSS. Similar pathological findings as in Zellweger syndrome also occur in other metabolic or hereditary CNS disorders (leukodystrophies) such as adrenoleukodystrophy, vanishing white matter disease and others. Laquinimod is a therapeutic approach in these otherwise untreatable CNS conditions.

### REFERENCES

Aubourg P, Wanders R (2013) Peroxisomal disorders. Handbook of clinical neurology 113:1593-1609.
Baes M, Gressens P, Baumgart E, Carmeliet P, Casteels M, Fransen M, Evrard P, Fahimi D, Declercq P E, Collen D, van Veldhoven P P, Mannaerts G P (1997) A mouse model for Zellweger syndrome. Nature genetics 17:49-57.
Baes M, Van Veldhoven P P (2012) Mouse models for peroxisome biogenesis defects and beta-oxidation enzyme deficiencies. Biochimica et biophysica acta 1822:1489-1500.
Bruck W, Pfortner R, Pham T, Zhang J, Hayardeny L, Piryatinsky V, Hanisch U K, Regen T, van Rossum D, Brakelmann L, Hagemeier K, Kuhlmann T, Stadelmann C, John G R, Kramann N, Wegner C (2012) Reduced astrocytic N F-kappaB activation by laquinimod protects from cuprizone-induced demyelination. Acta neuropathologica 124:411-424.
Kassmann C M, Lappe-Siefke C, Baes M, Brugger B, Mildner A, Werner H B, Natt O, Michaelis T, Prinz M, Frahm J, Nave K A (2007) Axonal loss and neuroinflammation caused by peroxisome-deficient oligodendrocytes. Nature genetics 39:969-976.
Mishra M K, Wang J, Keough M B, Fan Y, Silva C, Sloka S, Hayardeny L, Bruck W, Yong V W (2014) Laquinimod reduces neuroaxonal injury through inhibiting microglial activation. Annals of clinical and translational neurology 1:409-422.
Powers J M, Moser H W (1998) Peroxisomal disorders: genotype, phenotype, major neuropathologic lesions, and pathogenesis. Brain pathology 8:101-120.
Wanders R J, Poll-The B T (2015) “Role of peroxisomes in human lipid metabolism and its importance for neurological development”. Neuroscience letters.
Bottelbergs A. et al. (2010) “Axonal Integrity in the Absence of Functional Peroxisomes from Projection Neurons and Astrocytes”, GLIA, 56:1532-1543.
Crane D. I. (2014) “Revisiting the neuropathogenesis of Zellweger syndrome”, Neurochemistry International, 69:1-8.
Heiman G. et al. (2015) “Emerging Treatment for Pediatric Leukodystrophies”, Pediatric Clinics of North America, 62:649-666.
Kassmann C. M. et al. (2011) “A role for myelin-associated peroxisomes in maintaining paranodal loops and axonal integrity”, FEES Letters, 585=2205-2211.
Parikh S. et al. (2015) “A clinical approach to the diagnosis of patients with leukodystrophies and genetic leukoencephalopathies”, Molecular Genetics and Metabolism, 114:501-515.
Polman et al., (2005) “Treatment with laquinimod reduces development of active MRI lesions in relapsing MS”, Neurology. 64:987-991.25 RTT News Article dated April 12, II, entitled “Teva Pharma, Active Biotech Post Positive Laquinimod Phase 3 ALLEGRO Results”.
Sandberg-Wollheim et al. (2005) “48-week open safety study with high-dose oral laquinimod in patients”, Mult Scler. 11:S154 (Abstract).
Steinberg S. J. et al. (2006) “Peroxisome biogenesis disorders”, Biochimica et Biophysics Acta, 1763:1733-1748.

### Embodiments

1. A method of treating a subject suffering from leukodystrophy, the method comprising periodically administering to the subject an amount of laquinimod or pharmaceutically acceptable salt thereof effective to treat the subject.
2. The method of embodiment 1, wherein the pharmaceutically acceptable salt of laquinimod is laquinimod sodium.
3. The method of embodiments 1 or 2, wherein laquinimod is administered via oral administration.
4. The method of embodiments 1 or 2, wherein the laquinimod is administered in a composition comprising the laquinimod and an amount of an amino acid.
5. The method of embodiment 4, wherein the amino acid is selected from lysine, glycine, proline, alanine, or histidine.
6. The method of any one of embodiments 1-5, wherein administration is daily administration.
7. The method of any one of embodiments 1-5, wherein administration is more often than once daily.
8. The method of any one of embodiments 1-5, wherein administration is less often than once daily.
9. The method of any one of embodiments 1-8, wherein laquinimod administered is less than 0.6 mg/day.
10. The method of any one of embodiments 1-9, wherein laquinimod administered is 0.1-40.0 mg/day.
11. The method of any one of embodiments 1-10, wherein laquinimod administered is 0.1-2.5 mg/day.
12. The method of any one of embodiments 1-11, wherein laquinimod administered is 0.25-2.0 mg/day.
13. The method of any one of embodiments 1-12, wherein the amount laquinimod administered is 0.5-1.2 mg/day.
14. The method of embodiment 10, wherein the amount laquinimod administered is 0.25 mg/day, 0.3 mg/day, 0.5 mg/day, 0.6 mg/day, 1.0 mg/day, 1.2 mg/day, 1.5 mg/day, or 2.0 mg/day.
15. The method of any one of embodiments 1-14, wherein the periodic administration of laquinimod continues for at least 3 days, for more than 30 days, for more than 42 days, for 8 weeks or more, for at least 12 weeks, for at least 24 weeks, or for 6 months or more.
16. The method of any one of embodiments 1-15, wherein the subject is a human.
17. The method of any one of embodiments 1-16, wherein the leukodystrophy is 18q Syndrome, Acute Disseminated Encephalomyeolitis (ADEM), Acute Disseminated Leukoencephalitis, Acute Hemorrhagic Leukoencephalopathy, Adrenoleukodystrophy X-Linked (ALD), Adrenomyeloneuropathy (AM), Aicardi-Goutieres Syndrome, Alexander Disease, Adult-onset Autosomal Dominant Leukodystrophy (ADLD), Autosomal Dominant Diffuse Leukoencephalopathy (HDLS), Autosomal Dominant Late-Onset Leukoencephalopathy, Childhood Ataxia with diffuse CNS Hypomyelination (CACH or Vanishing White Matter Disease), Canavan Disease, Cerebral Autosomal Dominant Arteropathy with Subcortical Infarcts and Leukoencephalopathy (CADASIL), Cerebrotendinous Xanthomatosis (CTX), Craniometaphysical Dysplasia with Leukoencephalopathy, Cystic Leukoencephalopathy with RNASET2, Extensive Cerebral White Matter abnormality, Familial Adult-Onset Leukodystrophy, Familial Leukodystrophy, Globoid Cell Leukodystrophy (Krabbe Disease), Hereditary Adult Onset Leukodystrophy, Hypomyelination with Atrophy of the Basal Ganglia and Cerebellum (HABC), Hypomyelination, Hypogonadotropic, Hypogonadism and Hypodontia (4H Syndrome), Lipomembranous Osteodysplasia with Leukodystrophy (Nasu Disease), Metachromatic Leukodystrophy (MLD), Megalencephalic Leukodystrophy with subcortical Cysts (MLC), Neuroaxonal Leukoencephalopathy with axonal spheroids (Hereditary diffuse leukoencephalopathy with spheroids—HDLS), Neonatal Adrenoleukodystrophy (NALD), Oculodetatoldigital Dysplasia with cerebral white matter abnormalities, Orthochromatic Lleukodystrophy with pigmented glia, Ovarioleukodystrophy Syndrome, Pelizaeus Merzbacher Disease (X-linked spastic paraplegia), Refsum Disease, Sjogren-Larssen Syndrome, Sudanophilic Leukodystrophy, Van der Knaap Syndrome (Vacuolating Leukodystrophy with Subcortical Cysts or MLC), Vanishing White Matter Disease (VWM) or Childhood ataxia with diffuse central nervous system hypomyelination, (CACH), X-linked Adrenoleukodystrophy (X-ALD), Zellweger Syndrome, Neonatal Adrenoleukodystrophy, or Infantile Refsum Disease.
18. The method of any one of embodiments 1-16, wherein the leukodystrophy is a peroxisome biogenesis disorder.
19. The method of embodiment 18, wherein the peroxisome biogenesis disorder is Zellweger Syndrome, Neonatal Adrenoleukodystrophy, or Infantile Refsum Disease.
20. The method of embodiment 19, wherein the peroxisome biogenesis disorder is Zellweger Syndrome.
21. The method of any one of embodiments 1-20, wherein the amount of laquinimod is effective to reduce a symptom of leukodystrophy in the subject comparing to that in a subject afflicted with leukodystrophy not treated with laquinimod.
22. The method of embodiment 21, wherein the symptom is motor dysfunction, delayed development of motor skills, plateau in development of motor skills, regression in motor skills, rigidity, dystonia, ataxia, or bulbar symptoms.
23. The method of embodiment 21, wherein the symptom is cognitive impairment, developmental delay, intellectual disability, or dementia.
24. The method of embodiment 21, wherein the symptom is nystagmus, irritability, titubation, autonomic dysfunction, encephalopathy, endocrine dysfunction, ophthalmologic abnormalities, cortical visual impairment, dental abnormalities, dysmorphic physical features, tendinous xanthomas, skeletal abnormalities, bony abnormalities, hearing impairment, hepatosplenomegaly, cutaneous abnormalities, ovarian dysgenesis or gastrointestinal symptoms.
25. The method of embodiment 21, wherein the symptom is white matter abnormality.
26. The method of embodiment 25, wherein the white matter abnormality is demyelination, dysmyelination, or hypomyelination.
27. The method of embodiment 21, wherein the symptom is axonal loss.
28. The method of any one of embodiments 1-20, wherein the amount of laquinimod is effective to reduce a symptom of peroxisome biogenesis disorder (PBD) in the subject comparing to that in a subject afflicted with PBD not treated with laquinimod.
29. The method of embodiment 28, wherein the symptom of PBD is craniofacial abnormalities, high forehead, hypoplastic supraorbital ridges, epicanthal folds, midface hypoplasia, and large anterior fontanel, eye abnormalities, corneal clouding, cataracts, flaucoma, optic atrophy, retinal anomalies, neuronal migration defects, polymicrogyria, Purkinje cell heterotopia, olivary nucleus abnormities, hepatomegaly, renal cysts, chondrodysplasia punctate, hypotonia, seizures, inability to feed, impaired neonatal and deep tendon reflexes, impaired spontaneous movement, spasticity, sensorineural hearing loss, retinitis pigmentosa, disturbed ossification, shortening of the proximal long bones with metaphyseal cupping, coronal clefts of the vertebral bodies, epiphyseal stippling, contractures, bilateral cataracts, abnormal faces with frontal bossing, depressed nasal bridge, small nose, ichthyosis, central nervous system abnormalities, cerebral and cerebellar atrophy, abnormalities of myelination, neuronal migration defects, growth retardation, psychomotor retardation, respiratory complications, congenital cataracts, chondrodysplasia, rhizomelia, mental deficiency, growth deficiency, or learning disability.
30. The method of any one of embodiments 1-20, wherein the amount of laquinimod is effective to reduce a symptom of Zellweger syndrome (ZS) in the subject comparing to that in a subject afflicted with ZS not treated with laquinimod.
31. The method of embodiment 30, wherein the symptom of ZS is craniofacial abnormalities, high forehead, hypoplastic supraorbital ridges, epicanthal folds, midface hypoplasia, and large anterior fontanel, eye abnormalities, corneal clouding, cataracts, flaucoma, optic atrophy, retinal anomalies, neuronal migration defects, polymicrogyria, Purkinje cell heterotopia, olivary nucleus abnormities, hepatomegaly, renal cysts, chondrodysplasia punctate, hypotonia, seizures, inability to feed, impaired neonatal and deep tendon reflexes, or impaired spontaneous movement.
32. The method of any one of embodiments 1-20, wherein the amount of laquinimod is effective to reduce a symptom of Neonatal Adrenoleukodystrophy (NALD) in the subject comparing to that in a subject afflicted with NALD not treated with laquinimod.
33. The method of embodiment 32, wherein the symptom of NALD is craniofacial abnormalities, high forehead, hypoplastic supraorbital ridges, epicanthal folds, midface hypoplasia, and large anterior fontanel, eye abnormalities, corneal clouding, cataracts, flaucoma, optic atrophy, retinal anomalies, neuronal migration defects, polymicrogyria, Purkinje cell heterotopia, olivary nucleus abnormities, hepatomegaly, renal cysts, chondrodysplasia punctate, hypotonia, seizures, inability to feed, impaired neonatal and deep tendon reflexes, impaired spontaneous movement, spasticity, sensorineural hearing loss, or retinitis pigmentosa.
34. The method of any one of embodiments 1-20, wherein the amount of laquinimod is effective to reduce a symptom of Infantile Refsum Disease (IRD) in the subject comparing to that in a subject afflicted with IRD not treated with laquinimod.
35. The method of embodiment 34, wherein the symptom of IRD is craniofacial abnormalities, high forehead, hypoplastic supraorbital ridges, epicanthal folds, midface hypoplasia, and large anterior fontanel, eye abnormalities, corneal clouding, cataracts, flaucoma, optic atrophy, retinal anomalies, neuronal migration defects, polymicrogyria, Purkinje cell heterotopia, olivary nucleus abnormities, hepatomegaly, renal cysts, chondrodysplasia punctate, hypotonia, seizures, inability to feed, impaired neonatal and deep tendon reflexes, impaired spontaneous movement, spasticity, sensorineural hearing loss, or retinitis pigmentosa.
36. The method of any one of embodiments 21-35, wherein the symptom is reduced by at least 10%, at least 20%, at least 30%, at least 50%, at least 70%, more than 100%, more than 300%, or more than 1000%.
37. The method of any one of embodiments 1-36, wherein laquinimod is administered as an add-on therapy to or in combination with one or more other treatment for leukodystrophy.
38. The method of embodiment 37, wherein the other treatment for leukodystrophy is chenodeoxycholic acid, clofarabine, melphalan, alemtuzumab, mycophenolate mofetil, cyclosporine A, hydroxyurea, rabbit antithymocyte globulin, fludarabine, busulfan, cyclophosphamide, methylprednisolone, granulocyte colony-stimulating factor, granulocyte-macrophage colony-stimulating factor, N-acetylcysteine, celecoxib, vitamin E, alpha lipoic acid, campath-1H, cyclophosphamide, Lorenzo's oil, sobetirome, filgrastim, triheptanoim, glyceryl triacetate (GTA), chenodeoxycholic acid, lovastatin, betaine, and/or nutropin AQ.
39. Use of laquinimod in the manufacture of a medicament for treating a subject suffering from leukodystrophy.
40. Laquinimod for use in treating a subject suffering from leukodystrophy.
41. Use of laquinimod in treating a subject suffering from leukodystrophy.
42. A pharmaceutical composition comprising laquinimod for use in treating a subject suffering from leukodystrophy.
43. A pharmaceutical oral unit dosage form of laquinimod for use in treating a subject suffering from leukodystrophy.

1. A method of treating a subject afflicted with (a) leukodystrophy, (b) hereditary central nervous systems disorders, or (c) peroxisomal disorders, the method comprising periodically administering to the subject an amount of a compound of the general formula (I) 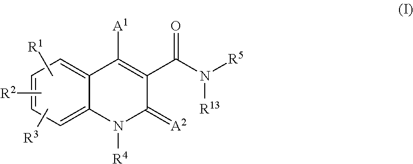
or a tautomer thereof wherein the groups A¹ and A² are interchanged and there is a 2,3-rather than a 3,4-double bond;
where R¹, R² and R³ are the same or different and are selected from the group consisting of: hydrogen; C₁-C₆ alkyl; C₁-C₆ alkenyl; C₁-C₅ alkoxy; C₁-C₆ alkylene; C₃-C₆ cycloalkyl; C₁-C₆ alkylthio; C₃-C₆ cycloakylthio; C₁-C₆ alkylsulfinyl; C₃-C₆ cycloalkylsulfinyl; aryl; acyl; heteroaryl; aralalkyl; allyl; carboxyl; amid; carbamoyl; carbonylamin; nitro; amino; cyano; trifluoromethyl; trifluoromethoxy; halogen; NO₂; OH; OCOR⁸; NR⁶R⁷; and NR⁶COR⁸; and where R¹ and R² or R² and R³ together may also be in the form of a methylenedioxy group;
where R⁴ is selected from the group consisting of: C₁-C₆ alkyl; C₁-C₆ alkenyl; C₁-C₅ alkoxy; C₁-C₆ alkylene; C₃-C₆ cycloalkyl; C₁-C₆ alkylthio; C₃-C₆ cycloakylthio; C₁-C₆ alkylsulfinyl; C₃-C₆ cycloalkylsulfinyl; aryl; acyl; heteroaryl; aralalkyl; allyl; carboxyl; amid; carbamoyl; carbonylamin; nitro; amino; cyano; C₁-C₆ alkylene forming a ring with the 8-position carbon atom of the quinoline ring system; cycloalkyl; optionally mono- or disubstituted; preferred optionally mono- or disubstituted with substituents selected from the group consisting of C₁-C₆ alkyl, C₁-C₆ alkoxy, OH and OCOR⁸; and phenyl, optionally mono- or disubstituted, preferred optionally mono- or disubstituted with substituents selected from the group consisting of C₁-C₆ alkyl, C₁-C₆ alkoxy and halogen;
and wherein R⁵ is selected from the group consisting of a five- or six-membered heterocyclic ring containing at most two heteroatoms selected from the group consisting of S and N, and being optionally mono- or disubstituted, preferred optionally mono- or disubstituted with substituents selected from the group consisting of C₁-C₆ alkyl, C₁-C₆ alkoxy, hydroxy and halogen; and wherein R⁵ may also be the group: 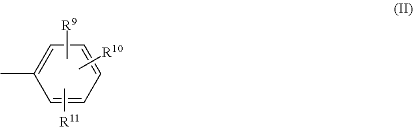
wherein R⁹, R¹⁰ and R¹¹ are the same or different and selected from the group consisting of: hydrogen; C₁-C₆ alkyl; C₁-C₆ alkenyl; C₁-C₅ alkoxy; C₁-C₆ alkylene; C₃-C₆ cycloalkyl; C₁-C₆ alkylthio; C₃-C₆ cycloakylthio; C₁-C₆ alkylsulfinyl; C₃-C₆ cycloalkylsulfinyl; aryl; acyl; heteroaryl; aralalkyl; allyl; carboxyl; amid; carbamoyl; carbonylamin; nitro; amino; trifluoromethyl; trifluoromethoxy; halogen; CN; SO₂CH₃; OH; OCOR⁶; NR⁶R⁷; NR⁶COR⁸; COOR¹²; OCH₂COOR¹²; CH₂COOR¹²; COR⁸; and 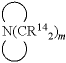
where each R¹⁴ are the same or different and are selected from the group consisting of: hydrogen; C₁-C₆ alkyl; C₁-C₆ alkenyl; C₁-C₅ alkoxy; C₁-C₆ alkylene; C₃-C₆ cycloalkyl; C₁-C₆ alkylthio; C₃-C₆ cycloakylthio; C₁-C₆ alkylsulfinyl; C₃-C₆ cycloalkylsulfinyl; aryl; acyl; heteroaryl; aralalkyl; allyl; carboxyl; amid; carbamoyl; carbonylamin; nitro; amino; cyano; trifluoromethyl; trifluoromethoxy; halogen; NO₂; OH; OCOR⁸; NR⁶R⁷; and NR⁶COR⁸ preferably wherein at least one of R¹⁴ are hydrogen;
wherein m is four or five; and where R⁹ and R¹⁰ or R¹⁰ and R¹¹ together also may be in the form of a methylenedioxy group;
wherein A¹ is selected from the group consisting of OR¹², OCOR⁸, NR⁶R⁷ and NR⁶COR⁸,
and wherein A² is selected from the group consisting of O and NR⁶;
wherein R⁶, R⁷ and R⁸ are the same or different and selected from the group consisting of hydrogen, C₁-C₆ alkyl; C₁-C₆ alkenyl; C₁-C₅ alkoxy; C₁-C₆ alkylene; C₃-C₆ cycloalkyl; C₁-C₆ alkylthio; C₃-C₆ cycloakylthio; C₁-C₆ alkylsulfinyl; C₃-C₆ cycloalkylsulfinyl; aryl; acyl; heteroaryl; aralalkyl; allyl; carboxyl; amid; carbamoyl; carbonylamin; nitro; amino; cyano;
wherein R¹² is selected from the group consisting of C₁-C₆ alkyl and M;
and wherein M is selected from the group consisting of hydrogen, O⁻ and pharmaceutically acceptable inorganic and organic cations;
and wherein R¹³ is selected from the group consisting of hydrogen, C₁-C₆ alkyl, C₁-C₆ alkenyl, C₁-C₅ alkoxy, C₁-C₆ alkylene, C₃-C₆ cycloalkyl, C₁-C₆ alkylthio, C₃-C₆ cycloakylthio, C₁-C₆ alkylsulfinyl, C₃-C₆ cycloalkylsulfinyl, aryl, acyl, heteroaryl, aralalkyl, allyl, carboxyl, amid, carbamoyl, carbonylamin, nitro, amino, cyano, preferred C₁-C₆ alkyl, optionally substituted with a substituent selected from the group consisting of OH, OR⁸ and OCOR⁸, and C₁-C₆ alkenyl;
provided that R¹³ is selected from the group consisting of C₁-C₆ alkyl, optionally substituted with a substituent selected from the group consisting of OH, OR⁸ and OCOR⁸, and C₁-C₆ alkenyl when R⁹, R¹⁰ and R¹¹ are selected from the group consisting of C₁-C₆ alkyl, C₁-C₆ alkenyl and C₁-C₆ alkoxy; and addition salts with pharmaceutically acceptable inorganic or organic acids.

2-15. (canceled)

16. The method of claim 1, wherein the subject is afflicted with leukodystrophy.

17. The method of claim 16 wherein the leukodystrophy selected from the group consisting of adrenoleukodystrophy, metachromatic leukodystrophy, globoid cell leukodystrophy (Morbus Krabbe), Pelizaeus-Merzbacher disease, Canavan-Syndrome, vanishing white matter leukoencephalopathy, Alexander disease, Refsum-Thiebaut disease, cerebrotendinous xanthomatosis, Morbus Batten and Zellweger Syndrome.

18. The method of claim 16 wherein the leukodystrophy is Zellweger Syndrome.

19. The method of claim 16 wherein R¹³ is selected from the group consisting of C₁-C₆ alkyl optionally substituted, A¹ is OH and A² is O, R⁴ is C₁-C₃ alkyl and R⁵ is group II.

20. The method of claim 16 wherein the compound is a compound of general formula (III) 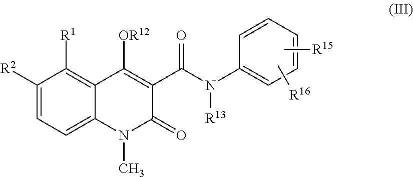
wherein
R¹³ is selected from methyl, ethyl, n-propyl, iso-propyl, n-butyl, iso-butyl, sec-butyl and allyl;
R¹² is selected from hydrogen and pharmaceutically acceptable inorganic and organic cations;
R¹ and R² are the same or different and selected from hydrogen, methyl, ethyl, n-propyl, iso-propyl, n-butyl, iso-butyl, sec-butyl, methoxy, ethoxy, chloro, bromo, CF₃, and OCH_{X}F_{Y};
wherein
x=0-2,
y=1-3 with the proviso that
x+y=3;
or
R¹ and R² taken together are methylenedioxy;
R¹⁵ is hydrogen, a straight or branched, saturated or unsaturated C₁-C₆-alkyl or -alkenyl, a cyclic C₃-C₆-alkyl, a straight or branched C₁-C₆-alkoxy, a cyclic C₃-C₆-alkoxy, fluoro, chloro, bromo, trifluoromethoxy or trifluoromethyl; and
R¹⁶ is hydrogen, fluoro or chloro, with the proviso that R¹⁵ is fluoro or chloro only when R¹⁶ is fluoro or chloro and any tautomer thereof.

21. The method of claim 16 wherein the compound is a compound of general formula (IV) 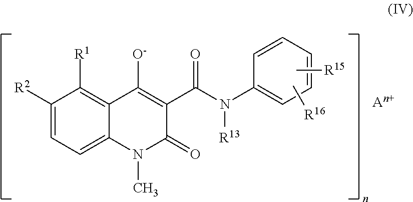
wherein
n is an integer of 1, 2 or 3;
Aⁿ+ is a mono- or multivalent metal cation selected from Li⁺, Na⁺, K⁺, Mg²⁺, Ca²⁺, Mn²⁺, Cu²⁺, Zn²⁺, Al³⁺ and Fe³⁺;
R¹³ is a straight or branched C₁-C₄-alkyl or -alkenyl or a cyclic C₃-C₄-alkyl;
R¹ and R² are the same or different and selected from hydrogen, straight or branched, saturated or unsaturated C₁-C₆-alkyl or -alkenyl, a cyclic C₃-C₆-alkyl, a straight or branched C₁-C₆-alkylthio, a cyclic C₃-C₆-alkylthio, a straight or branched C₁-C₆-alkylsulfinyl, a cyclic C₃-C₆-alkylsulfinyl, fluoro, chloro, bromo, trifluoromethyl or trifluoromethoxy; and/or
R¹ and R² taken together are methylenedioxy;
R¹⁵ is hydrogen, a straight or branched, saturated or unsaturated C₁-C₄-alkyl or -alkenyl, a cyclic C₃-C₄-alkyl, a straight or branched C₁-C₄-alkoxy, a cyclic C₃-C₄-alkoxy, fluoro, chloro, bromo or trifluoromethyl; and
R¹⁶ is hydrogen, fluoro or chloro, with the proviso that R¹⁶ is fluoro or chloro only when R¹⁵ is fluoro or chloro;
optionally, an alkaline-reacting component maintaining the pH preferably above 8, or a salt with a divalent metal cation.

22. The method of claim 16 wherein the compound is selected from the group consisting of Roquinimex (4-hydroxy-N, 1-dimethyl-2-oxo-N-phenyl-1,2-dihydroquinoline-3-carboxamide) and Laquinimod (5-chloro-N-ethyl-4-hydroxy-1-methyl-2-oxo-N-phenyl-1,2-dihydroquinoline-3-carboxamide).

23. The method of claim 16 wherein the compound is laquinimod.

24. A method of claim 1 wherein the subject is afflicted with a hereditary central nervous systems disorder.

25. The method of claim 24, wherein the compound is a compound of general formula (III) 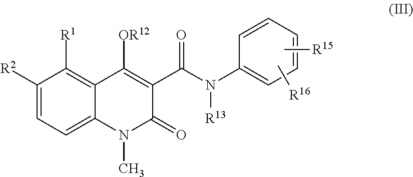
wherein
R¹³ is selected from methyl, ethyl, n-propyl, iso-propyl, n-butyl, iso-butyl, sec-butyl and allyl;
R¹² is selected from hydrogen and pharmaceutically acceptable inorganic and organic cations;
R¹ and R² are the same or different and selected from hydrogen, methyl, ethyl, n-propyl, iso-propyl, n-butyl, iso-butyl, sec-butyl, methoxy, ethoxy, chloro, bromo, CF₃, and OCH_{X}F_{Y};
wherein
x=0-2,
y=1-3 with the proviso that
x+y=3;
or
R¹ and R² taken together are methylenedioxy;
R¹⁵ is hydrogen, a straight or branched, saturated or unsaturated C₁-C₆-alkyl or -alkenyl, a cyclic C₃-C₆-alkyl, a straight or branched C₁-C₆-alkoxy, a cyclic C₃-C₆-alkoxy, fluoro, chloro, bromo, trifluoromethoxy or trifluoromethyl; and
R¹⁶ is hydrogen, fluoro or chloro, with the proviso that R¹⁵ is fluoro or chloro only when R¹⁶ is fluoro or chloro and any tautomer thereof.

26. The method of claim 24 wherein the compound is a compound of general formula (IV) 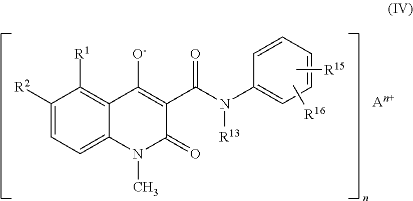
wherein
n is an integer of 1, 2 or 3;
Aⁿ+ is a mono- or multivalent metal cation selected from Li⁺, Na⁺, K⁺, Mg²⁺, Ca²⁺, Mn²⁺, Cu²⁺, Zn²⁺, Al³⁺ and Fe³⁺;
R¹³ is a straight or branched C₁-C₄-alkyl or -alkenyl or a cyclic C₃-C₄-alkyl;
R¹ and R² are the same or different and selected from hydrogen, straight or branched, saturated or unsaturated C₁-C₆-alkyl or -alkenyl, a cyclic C₃-C₆-alkyl, a straight or branched C₁-C₆-alkylthio, a cyclic C₃-C₆-alkylthio, a straight or branched C₁-C₆-alkylsulfinyl, a cyclic C₃-C₆-alkylsulfinyl, fluoro, chloro, bromo, trifluoromethyl or trifluoromethoxy; and/or
R¹ and R² taken together are methylenedioxy;
R¹⁵ is hydrogen, a straight or branched, saturated or unsaturated C₁-C₄-alkyl or -alkenyl, a cyclic C₃-C₄-alkyl, a straight or branched C₁-C₄-alkoxy, a cyclic C₃-C₄-alkoxy, fluoro, chloro, bromo or trifluoromethyl; and
R¹⁶ is hydrogen, fluoro or chloro, with the proviso that R¹⁶ is fluoro or chloro only when R¹⁵ is fluoro or chloro;
optionally, an alkaline-reacting component maintaining the pH preferably above 8, or a salt with a divalent metal cation.

27. The method of claim 24 wherein the compound is selected from the group consisting of Roquinimex (4-hydroxy-N, 1-dimethyl-2-oxo-N-phenyl-1,2-dihydroquinoline-3-carboxamide) and Laquinimod (5-chloro-N-ethyl-4-hydroxy-1-methyl-2-oxo-N-phenyl-1,2-dihydroquinoline-3-carboxamide).

28. The method of claim 24 wherein the compound is laquinimod.

29. A method of claim 1 wherein the subject is afflicted with a peroxisomal disorder.

30. The method of claim 29, wherein the compound is a compound of general formula (III) 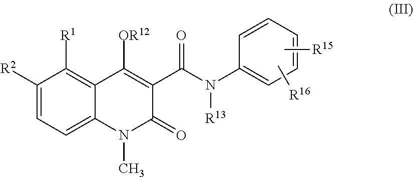
wherein
R¹³ is selected from methyl, ethyl, n-propyl, iso-propyl, n-butyl, iso-butyl, sec-butyl and allyl;
R¹² is selected from hydrogen and pharmaceutically acceptable inorganic and organic cations;
R¹ and R² are the same or different and selected from hydrogen, methyl, ethyl, n-propyl, iso-propyl, n-butyl, iso-butyl, sec-butyl, methoxy, ethoxy, chloro, bromo, CF₃, and OCH_{X}F_{Y};
wherein
x=0-2,
y=1-3 with the proviso that
x+y=3;
or
R¹ and R² taken together are methylenedioxy;
R¹⁵ is hydrogen, a straight or branched, saturated or unsaturated C₁-C₆-alkyl or -alkenyl, a cyclic C₃-C₆-alkyl, a straight or branched C₁-C₆-alkoxy, a cyclic C₃-C₆-alkoxy, fluoro, chloro, bromo, trifluoromethoxy or trifluoromethyl; and
R¹⁶ is hydrogen, fluoro or chloro, with the proviso that R¹⁵ is fluoro or chloro only when R¹⁶ is fluoro or chloro and any tautomer thereof.

31. The method of claim 29 wherein the compound is a compound of general formula (IV) 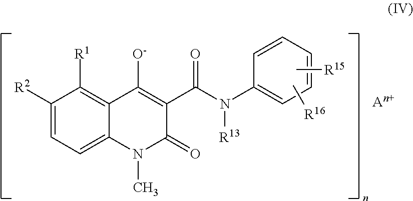
wherein
n is an integer of 1, 2 or 3;
Aⁿ+ is a mono- or multivalent metal cation selected from
Li⁺, Na⁺, K⁺, Mg²⁺, Ca²⁺, Mn²⁺, Cu²⁺, Zn²⁺, Al³⁺ and Fe³⁺;
R¹³ is a straight or branched C₁-C₄-alkyl or -alkenyl or a cyclic C₃-C₄-alkyl;
R¹ and R² are the same or different and selected from hydrogen, straight or branched, saturated or unsaturated C₁-C₆-alkyl or -alkenyl, a cyclic C₃-C₆-alkyl, a straight or branched C₁-C₆-alkylthio, a cyclic C₃-C₆-alkylthio, a straight or branched C₁-C₆-alkylsulfinyl, a cyclic C₃-C₆-alkylsulfinyl, fluoro, chloro, bromo, trifluoromethyl or trifluoromethoxy; and/or
R¹ and R² taken together are methylenedioxy;
R¹⁵ is hydrogen, a straight or branched, saturated or unsaturated C₁-C₄-alkyl or -alkenyl, a cyclic C₃-C₄-alkyl, a straight or branched C₁-C₄-alkoxy, a cyclic C₃-C₄-alkoxy, fluoro, chloro, bromo or trifluoromethyl; and
R¹⁶ is hydrogen, fluoro or chloro, with the proviso that R¹⁶ is fluoro or chloro only when R¹⁵ is fluoro or chloro;
optionally, an alkaline-reacting component maintaining the pH preferably above 8, or a salt with a divalent metal cation.

32. The method of claim 29 wherein the compound is selected from the group consisting of Roquinimex (4-hydroxy-N, 1-dimethyl-2-oxo-N-phenyl-1,2-dihydroquinoline-3-carboxamide) and Laquinimod (5-chloro-N-ethyl-4-hydroxy-1-methyl-2-oxo-N-phenyl-1,2-dihydroquinoline-3-carboxamide).

33. The method of claim 29 wherein the compound is laquinimod.

34. The method of claim 1 wherein the compound is orally administered to the patient.
